(19) United States
(12) Plant Patent Application Publication
(10) Pub. No.: US 2003/0121078 P1
(43) Pub. Date: Jun. 26, 2003

(54) SHRUB ROSE PLANT NAMED 'AUSQUEST'

(75) Inventor: David C.H. Austin, Albrighton (GB)

Correspondence Address: KLARQUIST SPARKMAN, LLP, One World Trade Center, Suite 1600, 121 S.W. Salmon Street, Portland, OR, 97204, US

(73) Assignee: David Austin Roses Limited

(21) Appl. No.: 10/032,268

(22) Filed: Dec. 20, 2001

(51) Int. Cl. A01H 5/00

(52) U.S. Cl. PLT/102

(57) ABSTRACT

Shrub rose plant having a robust and very branching growth habit; large, rosette-shaped flowers, cupped at first, peals reflexing later having a soft apricot color, paling to pale cream on outer petals; and freely produced flowers at the ends of slightly arching stems.

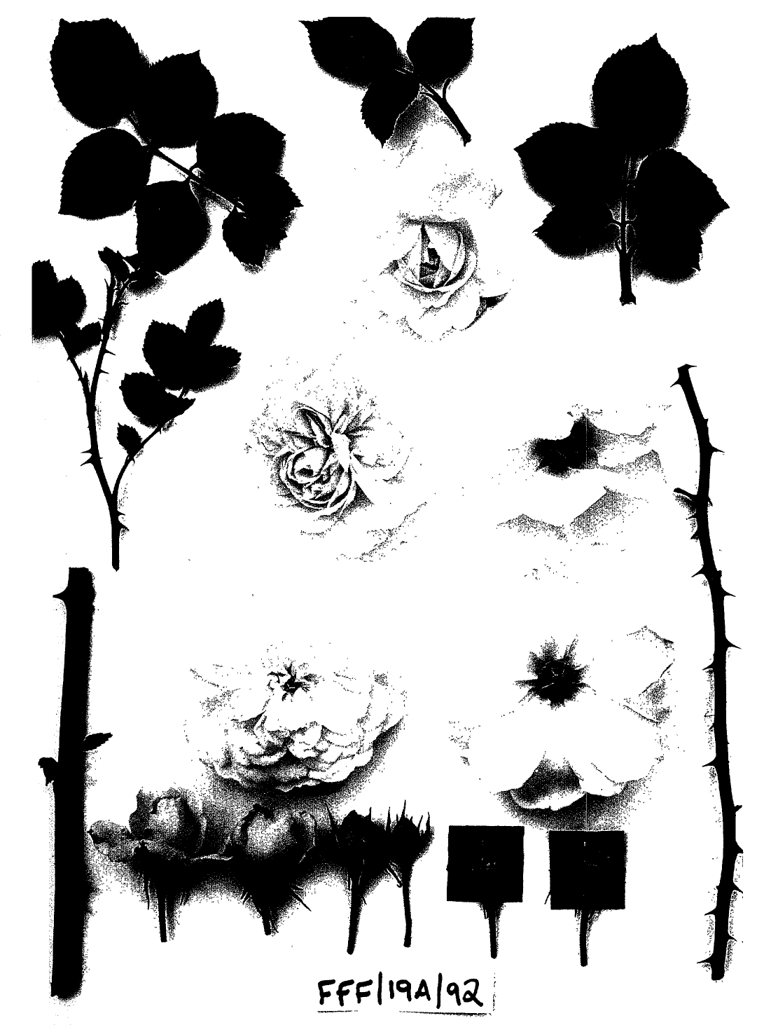

### SUMMARY OF THE INVENTION

The present invention relates to a new and distinct variety of rose plant of the shrub class which was originated by me by crossing an unnamed seedling, with the variety AUSgold, (U.S. Plant Pat. No. 8,688).

### BACKGROUND OF THE INVENTION

The primary objective of this breeding was to produce a new rose variety having a very free-flowering and healthy plant with flowers of the classic English rose shape and with a good fragrance.

### BRIEF SUMMARY OF THE INVENTION

The objective was substantially achieved, along with other desirable improvements, as evidenced by the following unique combination of characteristics that are outstanding in the new variety and that distinguish it from its parents, as well as from all other varieties of which I am aware:

1. Robust and very branching shrub;

2. Large, rosette-shaped flowers, cupped at first, petals reflexing later;

3. Soft apricot color, paling to pale cream on outer petals; and

4. Freely produced flowers at the end of slightly arching stems.

Asexual reproduction of this new variety by budding on Rosa dumetorum laxa rootstock, as performed at Albrighton, England, shows that the foregoing and all other characteristics and distinctions come true to form and are established and transmitted through succeeding propagations.

### COMPARISON WITH PARENTS

AUSquest differs from its seed parent in that the latter has rose pink flowers compared to AUSquest's apricot colored flowers and differs from its pollen parent which has rounded flowers as compared to AUSquest's rosette-shaped flowers.

### COMPARISON WITH SIMILAR VARIETY

AUSquest differs from AUSbaker, the most similar variety, in that AUSquest is broad and bushy with medium green foliage whereas AUSbaker has a large, arching growth habit and has dark green foliage.

### BRIEF DESCRIPTION OF THE ILLUSTRATION

The accompanying illustration shows typical specimens of the vegetative growth and flowers of this new variety in different stages of development, depicted in color as nearly true as it is reasonably possible to make the same in a color illustration of this character.

### DETAILED BOTANICAL DESCRIPTION

The following is a detailed description of my new rose cultivar with color descriptions using terminology in accordance with The Royal Horticultural Society (London) colour chart, except where ordinary dictionary significance of color is indicated.

Parentage:

Seed parent.—Unnamed seedling (unpatented).

Pollen parent.—AUSgold (U.S. Plant Pat. No. 8,688).

Classification:

Botanical.—Rosa hybrida.

Commercial.—Shrub.

### FLOWER

Observations made from plants of 6 years of age grown in a garden environment at Albrighton, England.

Blooming Habit: Recurrent.

Bud:

Size.—About 0.4 inches long when the petals start to unfurl.

Form.—The bud form is globular.

Color.—When sepals first divide, bud color is Yellow Group 4D. When half blown, the upper sides of the petals are White Group 155B; and the lower sides of the petals are Yellow Group 4D.

Sepals.—Color: Green Group 143A. Length: 0.8 inches. Width: 0.25 inch. Shape: Narrows to point. Surface texture: Serrated edges on appendaged sepals. There are three lightly appendaged sepals. There are two unappendaged sepals with hairy edges.

Receptacle.—Color: Yellow-Green Group 144A. Shape: Funnel. Size: Small. Surface: Smooth.

Peduncle.—Length: Medium, averaging about 1.6-2.4 inches. Surface: Prickly. Color: Yellow-Green Group 144B. Strength: Erect.

Bloom:

Size.—Medium. Average open size is diameter is about 3.2 inches.

Borne.—Singly and in small, irregular clusters of up to four blooms.

Stems.—Strength: Arching. Average length is about 28-32 inches.

Form.—When first open: Globular with outer petals curled back. Permanence: Outer petals curl back.

Petalage.—Number of petals under normal conditions: 95-110.

Color.—The upper sides of the petals are White Group 155B. The reverse sides of the petals are White Group 155D. The major color on the upper side is White Group 155B.

Variegations.—None.

Discoloration.—The general tonality at the end of the first day: Yellow-White Group 158B. As bloom ages color deepens to Yellow Group 10D with reddish spots.

Fragrance.—Moderate, with tea rose characteristics.

Petals:

Texture.—Thin, average.

Shape.—Circular.

Margin.—Slight frill.

Apex shape.—Rounded.

Base shape.—Goes to point.

Form.—Tips of outer petals slightly recurved, center petal edges slightly quill.

Arrangement.—Irregular.

Petaloids.—Large; lemon colored.

Persistence.—Petals hang on and dry.

Lastingness.—On the plant: Long. As a cut flower: Not tested.

Reproductive Parts: Anthers: Color: Yellow Group 12A. Arrangement: Tucked in calyx or partly so, mixed with petaloids. Filaments: Color: Yellow Group 4B. Pollen: Color: Yellow Group 8A.

Pistils.—Number: 40. Styles: Color: Green-Yellow Group 1C. Length: ½ inch. Stigmas: Color: Yellow Group 8A. Length: 1/32 inch.

Hips.—None produced.

### PLANT

Form.—Shrub, English rose type.

Growth.—Vigorous.

Mature plant.—Height: 4 feet. Width: 3 feet.

Rootstock.—recommended for United States.

Foliage: Number of leaflets on normal mid-stem leaves: 5-7.

Size.—Medium (4.5 inches long×5.6 inches wide).

Quantity.—Normal.

Color.—New foliage: Upper side: Green Group 137B, red tint on edges. Lower side: Green Group 137D. Old foliage: Upper side: Green Group 139A. Lower side: Green Group 137A.

Leaflets:

Size.—About 2 inches long×1.6 inches wide.

Shape.—Round and pointed (cuspidate).

Tip shape.—Pointed.

Apex shape.—Rounded.

Texture.—Semi-glossy.

Edge.—Serrated.

Serrations.—Small.

Petiole color.—Yellow-Green Group 146B.

Petiole rachis.—Color: Yellow-Green Group 144B. Underside: With prickles.

Stipules.—Length: 0.8 inches and serrated.

Auricle shape, if present.—Pointed.

Disease resistance.—Resistant to mildew, blackspot and rust under normal growing conditions.

Pest persistence.—Not tested.

Wood:

New wood.—Color: Yellow-Green Group 144B. Bark: Smooth.

Old wood.—Color: Yellow-Green Group 144A. Bark: Smooth.

Prickles:

Quantity.—On main canes from base: Ordinary. On laterals from main canes: Ordinary.

Form.—Hooked downward.

Length.—0.3 inch.

Color when young.—Yellow-Green Group 144C. Tip is Greyed-Purple Group 187C.

Color when mature.—Greyed-Red Group 181A.

Small Prickles:

Quantity.—On main stalks: None. On laterals: None.

Climate Hardiness: Hardy in Zones 4 to 9.

I claim:

1. A new and distinct variety of rose plant of the shrub class, substantially as herein shown and described, characterized particularly by its robust and very branching growth habit; large, rosette-shaped flowers, cupped at first, petals reflexing later, having a soft apricot color, paling to pale cream on outer petals; and freely produced flowers at the ends of slightly arching stems.
